(19) United States
(12) Patent Application Publication
(10) Pub. No.: US 2014/0039315 A1
(43) Pub. Date: Feb. 6, 2014

(54) Medical devices with visibility-enhancing features

(71) Applicant: Baylis Medical Company Inc., Mississauga (CA)

(72) Inventors: Gareth Davies, Toronto (CA); John Paul Urbanski, Toronto (CA); Eric Ma, Toronto (CA)

(73) Assignee: Baylis Medical Company Inc., Mississauga (CA)

(21) Appl. No.: 13/962,396

(22) Filed: Aug. 8, 2013

Related U.S. Application Data
(63) Continuation-in-part of application No. 13/468,939, filed on May 10, 2012.
Application No. 13/468,939 is a division of application No. 11/905,447, filed on Oct. 1, 2007, now Pat. No. 8,192,425.
(60) Provisional application No. 61/681,512, filed on Aug. 9, 2012.
(60) Provisional application No. 60/827,452, filed on Sep. 29, 2006.
(60) Provisional application No. 60/884,285, filed on Jan. 10, 2007.

(51) Int. Cl. A61M 25/01; A61B 8/08

(52) U.S. Cl. CPC A61M 25/0108; A61B 8/0841
USPC 600/439; 604/528

(57) ABSTRACT

A method and apparatus are disclosed for a medical device, including a means for enhancing visibility under imaging which, while reducing the local mechanical strength, avoids compromising the overall device effectiveness or safety. Visibility under imaging is increased by a non-uniform distribution of visibility-enhancing features as a function of the local stresses expected during use. One embodiment is for a medical device comprising an elongate member having proximal and distal regions, wherein the distal region comprises: a first echogenic region including a plurality of circumferential first region cuts, each of the first region cuts having a first region cut volume; and a second echogenic region, distal of the first echogenic region, including a plurality of circumferential second region cuts, wherein each of the second region cuts has a second region cut volume greater than said first region cut volume.

### REFERENCES TO PARENT AND CO-PENDING APPLICATIONS

This application claims the benefit of the filing date of U.S. Provisional Application Ser. No. 61/681,512, filed Aug. 9, 2012, entitled “Medical devices with visibility-enhancing features,” the entire disclosure of which is hereby incorporated by reference into the present disclosure. This application is also a Continuation-in-part of and claims priority to U.S. patent application Ser. No. 13/468,939, which is a divisional application of, and claims priority from, U.S. application Ser. No. 11/905,447, filed on Oct. 1, 2007, now U.S. Pat. No. 8,192,425, which claims the benefit of: U.S. provisional application No. 60/827,452, filed on Sep. 29, 2006, and U.S. provisional application No. 60/884,285, filed on Jan. 10, 2007, all of which are incorporated by reference herein in their entirety.

### TECHNICAL FIELD

The disclosure relates to medical devices with visibility-enhancing features. More specifically, the disclosure relates to the distribution and characteristics of such visibility-enhancing features.

### SUMMARY OF THE DISCLOSURE

In one broad aspect, embodiments of the present invention include a medical device comprising an elongate member having proximal and distal regions, the distal region comprising: a first echogenic region including a plurality of circumferential first region cuts, each of the first region cuts having a first region cut volume; and a second echogenic region including a plurality of circumferential second region cuts, each of the second region cuts having a second region cut volume greater than said first region cut volume; wherein the second echogenic region is located at a portion of the distal region subject to less bending stress in use, relative to a location of the first echogenic region.

As a feature of the first broad, the elongate member has a circular cross-section and is operable to be inserted through a dilator when in use. The elongate member is configured such that, in use, the second echogenic region is located further distal than the first echogenic region relative to an end of the dilator through which the elongate member is inserted.

As another feature of the first broad aspect, mechanical integrity of the elongate member under stress is substantially equivalent along its length.

As another feature of the first broad aspect, a second region cut density is greater than a first region cut density i.e. the second region has a higher number of cuts per unit of area than the first area.

In another broad aspect, embodiments of the present invention include a medical device comprising an elongate member having proximal and distal regions, the distal region comprising: a first echogenic region including a plurality of circumferential first region cuts, each of the first region cuts having a first region cut volume; and a second echogenic region, distal of the first echogenic region, including a plurality of circumferential second region cuts, each of the second region cuts having a second region cut volume greater than said first region cut volume.

In yet another broad aspect, embodiments of the present invention are for a method of creating a perforation of tissue within a heart, the method comprising: inserting an elongate medical device into a heart of a patient's body; visualizing one or more echogenic markings associated with the medical device to facilitate positioning of the medical device at a target tissue site within the heart; and delivering energy to a distal region of the elongate medical device to create a perforation through the target tissue site.

As a feature of the broad aspect, the target tissue site comprises a septum of a heart.

### BRIEF DESCRIPTION OF THE DRAWINGS

In order that the invention may be readily understood, embodiments of the invention are illustrated by way of examples in the accompanying drawings, in which:

FIG. 1 is a side view of the distal end of an embodiment of a medical device having cut out rings, with a lumen shown in broken line;

FIG. 2A is a cut away view of the embodiment of FIG. 1;

FIG. 2B is an embodiment similar to the cut away view embodiment of FIG. 2A with the addition of an end cap;

FIG. 3 is a perspective view of the embodiment of FIG. 1;

FIG. 4A is a side view of the embodiment of FIG. 2B projecting from a dilator;

FIG. 4B is a cut-away view of the embodiment of FIG. 4A and FIG. 4C is a line graph corresponding to FIG. 4B showing force and bending moment;

FIGS. 5A and 5B are illustrations of an embodiment of the invention in use;

FIG. 6 is a flow chart showing steps that may be performed in the development of embodiments of the present invention;

FIG. 7 is a side view of the distal end of an embodiment of a medical device having projecting rings;

FIG. 8 is a side view of the distal end of an embodiment of a medical device including a polymer with trapped bubbles;

FIG. 9 is a side view of the distal end of an embodiment of a medical device having structural or mechanical discontinuities, including a joint;

FIG. 10 is a side view of the distal end of an embodiment of a medical device with curved surfaces;

FIGS. 11A and 11B are diagrams relating to the bending stress of an elongate member (shaft) of a medical device;

FIG. 12 is a cut away view of the distal end of an embodiment of a medical device having a coating; and

FIG. 13 is a diagram illustrating angled cuts.

### DETAILED DESCRIPTION

Imaging systems utilized during the course of medical procedures include X-ray (fluoroscopy), ultrasound and magnetic resonance imaging (MRI). Examples of means to improve imaging include radiopaque markers and coatings in the case of fluoroscopy, magnetic components including magnetic coils for MRI, and surface markings in the case of ultrasound.

Ultrasound techniques include intracardiac echocardiography (ICE) and transesophageal echocardiography (TEE). A partial list of devices that may use ultrasound includes biopsy needles, tissue puncture devices (e.g. transseptal puncture devices), fluid collection devices, lesioning devices, devices requiring vascular access, in vitro fertilization devices, obstetrics and gynecology devices, and devices for tissue removal. To aid in the performance of medical procedures, it is desirable to increase the visibility of medical devices under imaging without compromising overall device efficacy or safety. To increase visibility under ultrasound, it is beneficial to have larger cuts into the surface of a medical device as echogenic performance tends to increase with depth, diameter, and density of surface voids. For example, deeper and wider rings may improve the effective viewing angles and the mount of signal that can be reflected back to an ultrasound sensor. However, as understood by one skilled in the art, cuts in the surface of a long shaft (an elongate member) subject to bending moments, can reduce the local mechanical integrity of the shaft at the location (i.e. region) of the cuts, thereby making the shaft more susceptible to breakage.

The present inventors have discovered a novel and unique means for enhancing visibility under imaging of a medical device which, while reducing mechanical strength locally, avoids compromising the overall device effectiveness or safety. The means include increasing visibility under imaging with a non-uniform distribution of visibility-enhancing features as a function of the local stresses expected during use. Embodiments of the devices provided by said means display increased visibility along the device both in areas of relatively greater mechanical vulnerability and lesser mechanical vulnerability, wherein the visibility is increased to a greater extent in areas of lesser mechanical vulnerability (expected stress) relative to areas of greater mechanical vulnerability (expected stress). Larger, denser, and ultimately more effective echogenic features are located in areas wherein low expected mechanical stress is expected in use. Conversely, in areas where higher stresses are expected in use, smaller features are used. Embodiments of the present invention provide enhanced visibility of medical devices while maintaining the structural integrity of the devices to a greater extent relative to other devices with similar visibility enhancing features.

Some embodiments of the invention incorporate one or more discrete surface irregularities, such as a single cut or groove, or a plurality of cuts or grooves, for imaging under ultrasound. As previously noted, deeper and wider cuts, in general, improve the effective viewing angles and the amount of signal that may be reflected back to an ultrasound sensor. An embodiment of the present invention displaying such features is a medical device comprising an elongate member having proximal and distal regions. The distal region comprises a first echogenic region including a plurality of circumferential first region cuts, each of the first region cuts having a first region cut volume, and a second echogenic region including a plurality of circumferential second region cuts, each of the second region cuts having a second region cut volume greater than said first region cut volume wherein the second echogenic region is located at a portion of the distal region subject to less bending stress in use, relative to a location of the first echogenic region. Such embodiments provide for increased echogenicity by having the surface cuts in the stronger regions of the device, thereby maintaining the overall mechanical integrity of a device. In some embodiments, the second region is distal of the first region; reasons for a distal region being under less bending stress than a relatively proximal region are explained hereinbelow.

The circumferential cuts of the above example are located around 360° of the circumference of the device (or for non-circular alternative embodiments, around substantially 360° of the perimeter of the device), whereby the device is substantially visible from all (radial) directions (that is, at any angle relative to a transducer).

In general, a visibility enhancing feature, in accordance with embodiments of the invention, is positioned in regions of the device located between structural discontinuities (points of weakness e.g. hinges, joints, welds, apertures). In the case of the above described example of a medical device, the circumferential cuts are located between structural weaknesses, rather than in the region of a structural discontinuity.

With specific reference now to the drawings in detail, it is stressed that the particulars shown are by way of example and for purposes of illustrative discussion of certain embodiments of the present invention only. Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

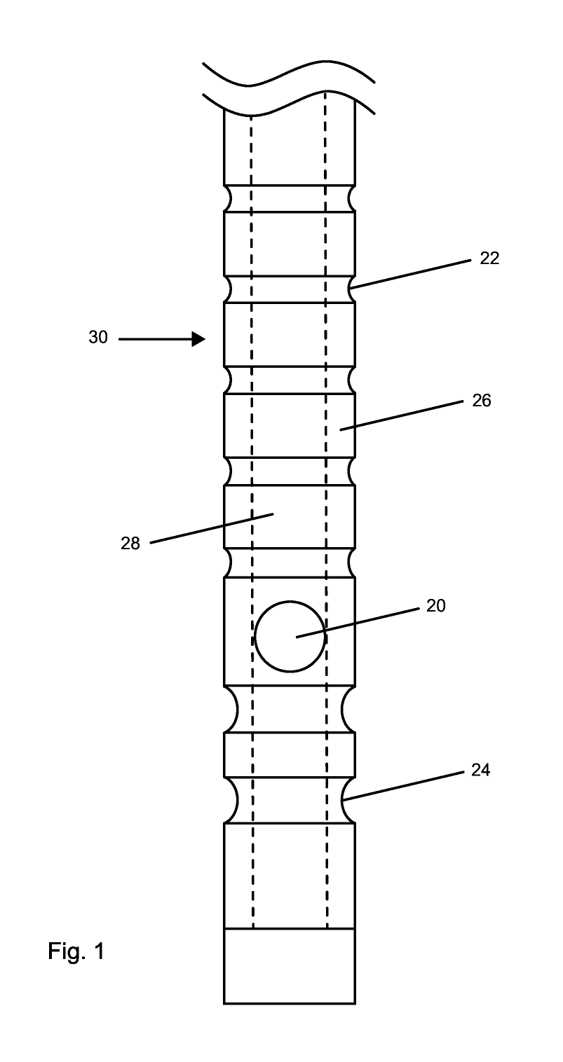

FIGS. 1 to 4 illustrate a medical device (e.g. a needle) that may be used in conjunction with ultrasound imaging.

FIG. 1 is a side view of the distal end of an embodiment of a medical device, such as a biopsy or radiofrequency needle, having cut-out rings (i.e. rounded grooves). The medical device 30 includes elongate member 26 (a shaft), lumen 28 (indicated by broken lines), a side-port aperture 20 that is in fluid communication with lumen 28, cut-out rings 22 proximal of aperture 20 and cut-out rings 24 distal of aperture 20. The volume of material removed to form rings 24 is greater than the volume removed to form rings 22. The rings increase the echogenicity of medical device 30, with relatively larger rings 24 typically providing more echogenicity that relatively smaller rings 22. The discrete elements of the visibility enhancing feature (in this case, each of the rings 22 and 24) are formed around substantially 360° of the circumference of the medical device 30 whereby medical device 30 may be visible from all directions (i.e. at any angle relative to an ultrasonic transducer). Some particular embodiments have a nominal (i.e. uncut and without insulation) wall thickness of about 0.15 mm and cut-out rings that are about 50 percent of the wall thickness located around 360° of the perimeter of the medical device. In some embodiments of the invention, such as the example of FIG. 1, the elongate member defines a lumen, while in alternative embodiments, it does not.

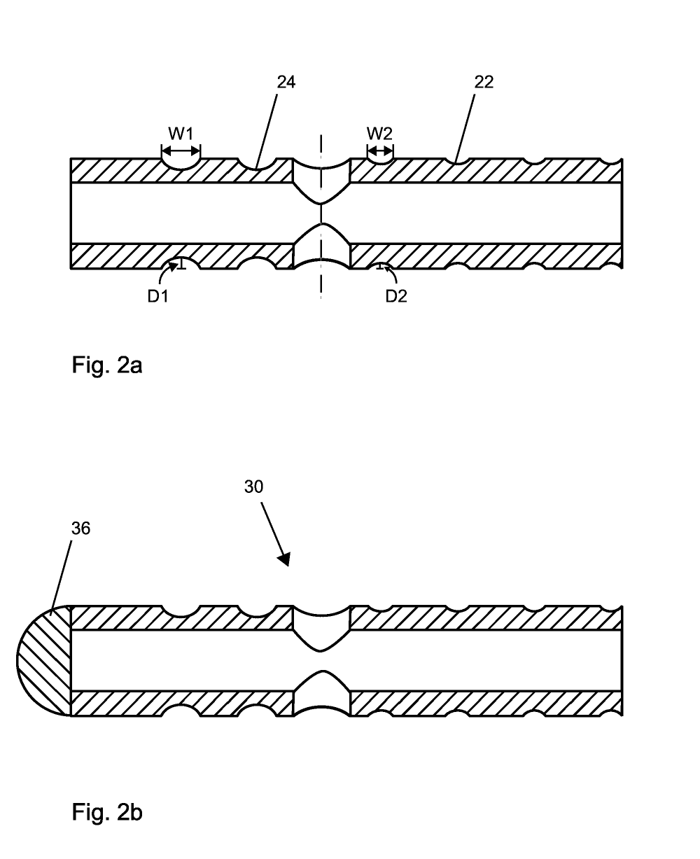

FIG. 2A is a cut away view of the embodiment of FIG. 1. Larger volume cut-out rings 24, have depth D1 and width W1 and smaller volume cut-out rings 22, have depth D2 and width W2, wherein D1>D2 and/or W1>W2. In some alternative embodiments W1=W2 while D1>D2, and in other alternative embodiments D1=D2 while W1>W2.

Medical device 30 of FIGS. 1 and 2A is an example of a medical device comprising an elongate member 26 having proximal and distal regions. The distal region includes first and second echogenic regions. The first echogenic region comprises a plurality of circumferential first region cuts (cut-out rings 22) with each of the first region cuts having a first region cut volume. The second echogenic region is distal of the first echogenic region and comprises a plurality of circumferential second region cuts (cut-out rings 24) wherein each of the second region cuts has a second region cut volume greater than the first region cut volume.

In a particular embodiment (without a coating of insulation), larger volume cut-out rings 24 have a width W1 of about 0.25±0.05 millimeters (mm) and a depth D1 of about 0.075 mm (with an upper deviation of 0.000 and a lower deviation of 0.025), and smaller volume cut-out rings 22 have a width of about 0.15±0.05 mm and a depth D1 of about 0.04 mm (with an upper deviation of 0.00 and a lower deviation of 0.02 mm). In some particular embodiments the medical device defines a lumen and a basic (nominal) wall thickness of about 0.15 mm. In some embodiments having a wall thickness of about 0.15 mm the depth of the larger volume cut-out rings 24 will be from about 33 to 50 percent of the wall thickness and the smaller volume cut-out rings 22 will be from about 13 to 27 percent of the wall thickness. In the illustrated embodiments, one or more of the individual components of the visibility enhancing feature (the cut rings) are located around 360° of the perimeter or circumference of the medical device. In alternative embodiments, the cut rings do not extend around the entire (i.e. the full 360°) perimeter or circumference. The rings may be produced using tooling of the appropriate size.

FIG. 2B is an embodiment similar to the cut away view embodiment of FIG. 2A. It has the additional feature of end cap 36. In some embodiments, end cap 36 is an electrode or other energy delivery structure or component.

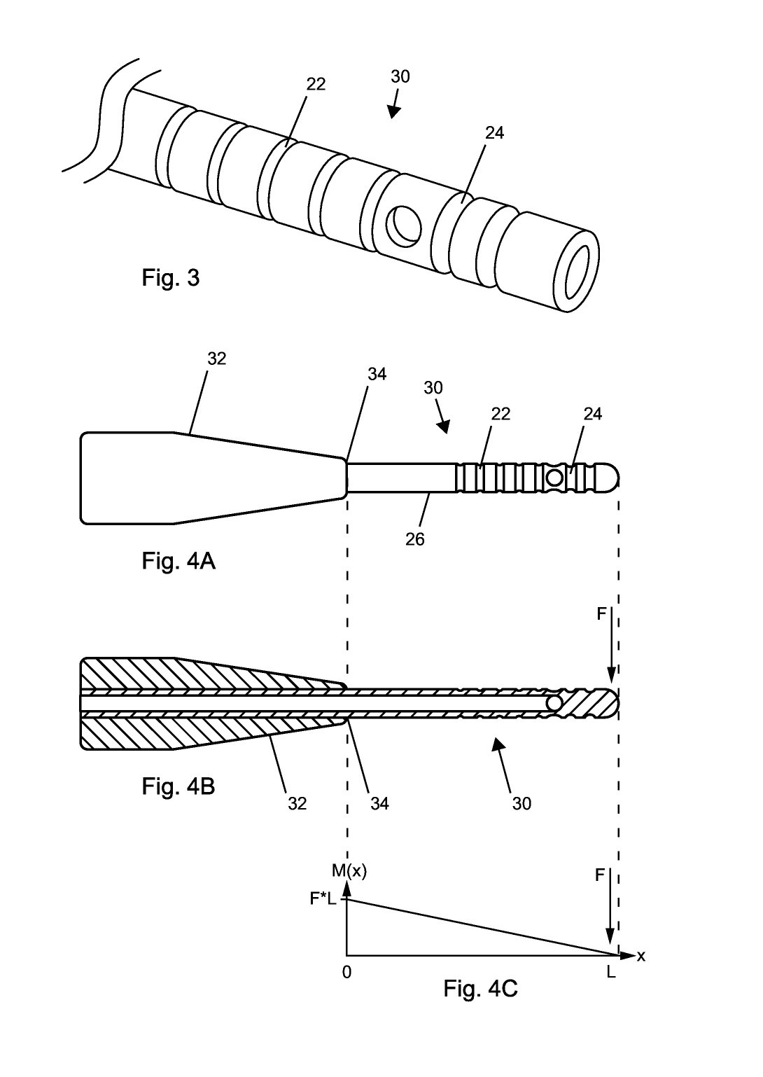

FIG. 3 is a perspective view of the embodiment of FIGS. 1 and 2A illustrating the various cut-out rings along the device.

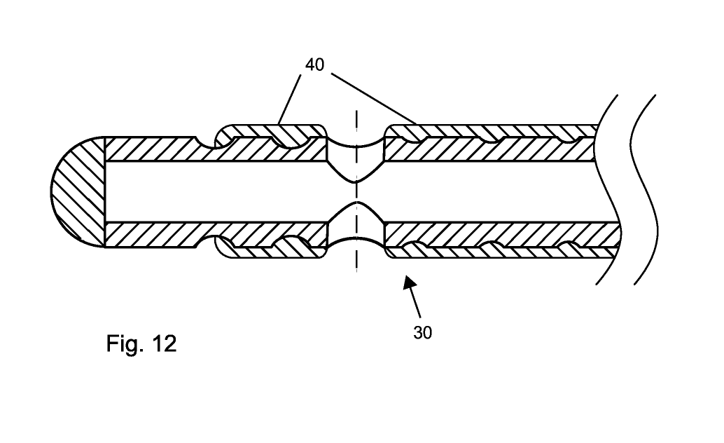

The embodiments shown in FIGS. 1 to 4 do not include a coating over the outer surface of the device. In alternative embodiments, the medical device may be covered with a coating, for example, a heat-shrink coating or other surface covering including a polymeric material such as Polytetrafluoroethylene (PTFE). The previously disclosed dimensions for wall thickness define for an elongate member 26 but not coatings thereupon. In some embodiments including a coating, the distal tip of the device (which may include an electrode, for example) is left exposed and in some particular embodiments, a polymer coating 40 may terminate in a cut-out groove, such as in the example of FIG. 12.

Differences in acoustic impedances between two materials may create an interface that will cause a portion of the ultrasonic pulsed beam to be reflected. The greater the acoustic mismatch, the greater will be the intensity of the reflected portion of the beam. Some embodiments of the medical device include a metal shaft, having relatively high acoustic impedance, covered by a coating having an acoustic impedance that facilitates the use of ultrasound for imaging wherein the coating has an acoustic impedance substantially similar to the acoustic impedance of blood (about 1.51 MRayl) and soft tissues (from about 1.30 MRayl for fat to about 1.7 MRayl for muscle). Due to this matching of acoustic impedance between the coating and the surrounding material (blood or soft tissue) when the device is in use in the patient's body, the interface between the coating and the soft tissue (or blood) will reflect a relatively small portion of an ultrasonic pulsed beam. This then allows a large portion of the ultrasonic beam to proceed through to the metal shaft, which has a relatively high acoustic impedance (e.g. Z=45.7 MRayl for stainless steel), whereby a large portion of the ultrasonic beam will be reflected by the metallic surface (or the interface between the coating and the shaft).

An example of such an embodiment may include a coating comprising a polymeric electrical insulation layer with acoustic impedance that substantially matches blood to thereby allow a majority of the ultrasonic wave through the blood-insulation interface in order to reflect off of the metal-insulation interface This acoustic impedance matching helps to provide an improved image relative to the image provided by a coating with an acoustic impedance outside the range of soft tissue and blood. Examples of suitable polymer coatings with suitable acoustic impedances include silicone rubber which may have an acoustic impedance of 1.40 MRayl, and DGEBA (epoxy resin) which may have an acoustic impedance of about 1.48 to about 1.53 MRayl.

FIG. 4A is a side view of the embodiment of FIG. 2B projecting from a dilator 32. In this specific case the dilator provides support to elongate member 26 of medical device 30 proximal of aperture 20. The dilator 32 and elongate member 26 (a needle shaft) may be described mechanically as a cantilevered system. In general, an elongate member supported at one of its ends, by for example, a handle, has a susceptibility to bending stress at the location of the support. In the configuration of FIG. 4A, medical device 30 is relatively more mechanically vulnerable about the distal end of dilator 32 at point 34. While the embodiment of FIG. 4A illustrates medical device 30 being supported by a dilator, alternative embodiments include using other proximal supports, for example, a handle.

FIG. 4B is a cut-away view of the embodiment of FIG. 4A and FIG. 4C is a line graph corresponding to FIG. 4B showing force and bending moment. For explanatory purposes, and as described above, the elongate member (a shaft) is considered to be fixed at its proximal end. The cantilevered portion of the elongate member is the portion of elongate member 26 of medical device 30 projecting a distance L from dilator 32. FIG. 4C illustrates an x-axis value of 0 at point 34 and a downward force F at the distal end of elongate member 26 of medical device 30 which causes the cantilevered elongate member to experience a bending moment. Assuming a single point force F at the distal end, it can be calculated (using M(x)=F·(L−x)) that the moment M experienced at the support (at point 34) is equal to the point force multiplied by the distance from the point of force application, i.e. M=F·L at point 34. As the length of the moment arm decreases towards the free tip of the elongate member, the bending moment also decreases, with the midpoint of the cantilevered elongate member experiencing a moment of FL/2. As understood by a person having ordinary skill in the art, in this configuration, stresses induced on the device through bending are much greater than those induced through shear or axial tension or compression.

Echogenic markings (rings, dimples, notches, etc.) in a medical device may reduce the mechanical integrity of the device (i.e. the ability of the device to withstand forces and stresses without permanently deforming or breaking), with markings produced by removing a larger amount of material causing larger reductions in mechanical integrity. Considering the embodiment of FIG. 4, if the medical device 30 is designed to have echogenic markings that are uniform in size and are sufficiently small so that the medical device can withstand a moment of FL along the entire length of the cantilevered elongate member, then the echogenic markings will necessarily be smaller than required at those locations of the elongate member that can be expected to experience bending moments smaller than FL. For example, the markings would be smaller than required at the midpoint of the cantilevered elongate member, which is expected to experience a moment of FL/2 (in the FIG. 4B configuration). In this particular example, typically, the effective depth (size) of the markings could be increased distally towards the tip of the cantilevered elongate member, inversely proportional to the stresses induced by the bending moment, such that that the mechanical integrity under stress will be substantially equivalent along the elongate member, i.e. no single marking location would represent the weakest position on the elongate member.

Referring to FIG. 4A, the region with cut-out rings 22 (proximal of the side-port aperture) is typically subject to more bending in use than the region with cut-out rings 24 (distal of the aperture), and the region without cut-out rings (closer to dilator 32 than cut-out rings 22) is subject to yet even more bending stress.

The medical device of FIG. 4a is an example of a medical device 30 comprising an elongate member 26 having proximal and distal regions, with the distal region comprising first and second echogenic regions. The first echogenic region includes a plurality of circumferential first region cuts (cut-out rings 22), each of the first region cuts having a first region cut volume. The second echogenic region includes a plurality of circumferential second region cuts (cut-out rings 24), each of the second region cuts having a second region cut volume which is greater than the first region cut volume, Also, the second echogenic region is located at a portion of the distal region subject to less bending stress in use, relative to the location of the first echogenic region.

The above example, using the formula M(x)=F·(L−x), represents a simplification, as not all forces are accounted for and the in-use position of the medical device (relative to the dilator) may vary. A different in-use position may require an analysis based on a different device position, a different length L, and a different force F magnitude or direction. Considering the different possibilities of normal use, a device may be designed by one skilled in the art to account for the maximum extension and force expected in normal use.

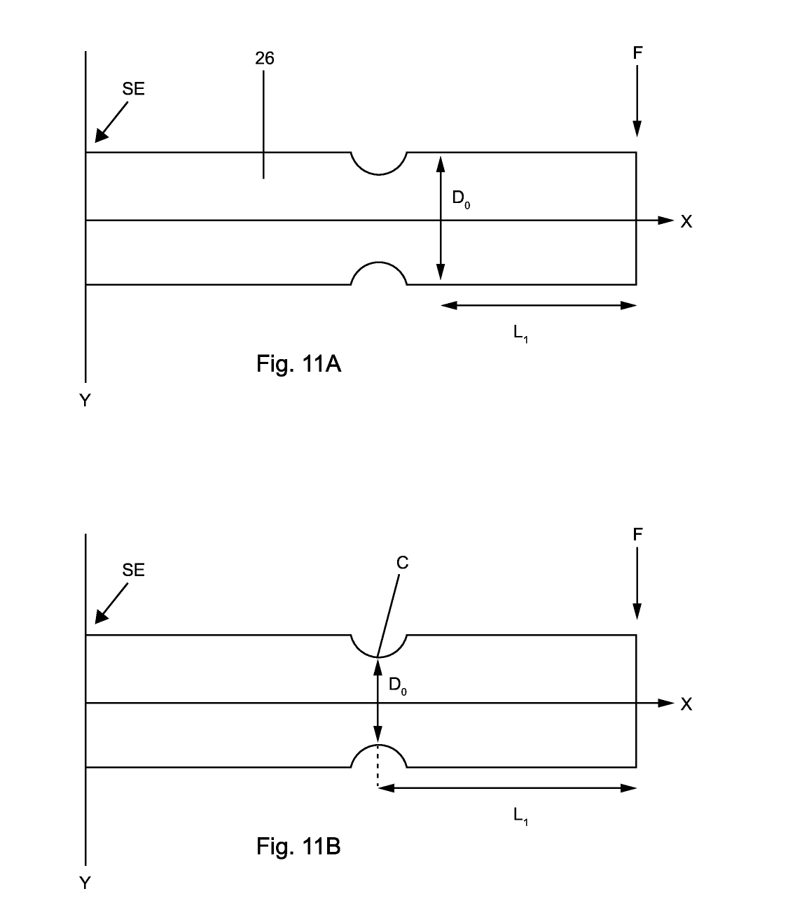

Making reference to FIGS. 11A and 11B, the significance of the cut depth may be appreciated by consideration of the formula:

$$\begin{matrix}σ = My / I \\ = ( ( F {1}_{1} ) ( {D}_{0} ( x ) / 2 ) ) / ( ( π / 64 ) {{D}_{0} ( x )}^{4} ) \\ = 32 F {1}_{1} / ( π {{D}_{0} ( x )}^{3} )\end{matrix}$$

wherein x is a point along a supported elongate member 26 (a shaft) of medical device 30, σ is bending stress, F is a downward/lateral force, I₁ is the distance from the end of the elongate member to x, and Dₒ(x) is the outer diameter of elongate member 26 at position x. Elongate member 26 is supported at support end SE. In this formula, the bending stress is proportional to the inverse of outer diameter Dₒ (x) to the third power. The above formula does not take into account all of the design features a medical device may have, and does not include all the forces that may act on a medical device. It may be simplified to express the approximation σ≈K/Dₒ(x)³ wherein K is a constant.

In the example of FIGS. 11A and 11B, the outer diameter Dₒ in FIG. 11A is taken at a point on elongate member 26 having a maximum outer diameter and the outer diameter Dₒ in FIG. 11B is taken at the smallest diameter, point C, at the location of a cut-out ring. Due to the bending stress being proportional to the inverse of outer diameter Dₒ (x) to the third power (σ≈K/Dₒ(x)³), the stress will be significantly higher at the location of the cut in FIG. 11B than at the larger outer diameter location of FIG. 11A. In other words, the approximation σ≈K/Dₒ(x)³ indicates that the stress will be less at the location along the elongate member (shaft) of the larger outer diameter Dₒ of FIG. 11A than at the location of the cut.

In order to represent the distribution of visibility enhancing features (which may also be referred to as the individual components of a visibility enhancing feature) along the device, a parameter referred to as an “echogenic feature scale” may be defined. The echogenic feature scale refers to the distribution, size and configuration of the cuts, grooves or other features along the device, which will typically be non-uniform and which is governed by and optimized in view of the stress expected at a given position (approximated by σ≈K/Dₒ(x)³) as well as the increase in visibility provided by the particular feature.

For example, in the case of a cut or groove, both the increase in visibility as well as the decrease in mechanical integrity, are proportional to the volume of the cut/groove. Therefore, if a given location along the device is expected to experience lower stresses, then less mechanical integrity is required and the cuts/grooves may be made deeper and/or wider than at other locations along the device in order to increase the visibility at that location. The same device may also include locations where greater stresses are expected, in which case the cuts/grooves at those locations may be made shallower and/or narrower relative to the cuts/grooves mentioned previously in order to avoid substantially reducing the mechanical integrity of the device at those locations. In this case, the “echogenic feature scale” may refer to the depth and/or width of the individual cuts/grooves, such that the echogenic feature scale may be described to be non-uniform along the device. In alternate embodiments, the echogenic feature scale may refer to any other feature or characteristic of the visibility enhancing feature or the individual components of the visibility enhancing feature, including but not limited to size (depth, width, etc.), shape and density (number of visibility enhancing feature(s) within a given area).

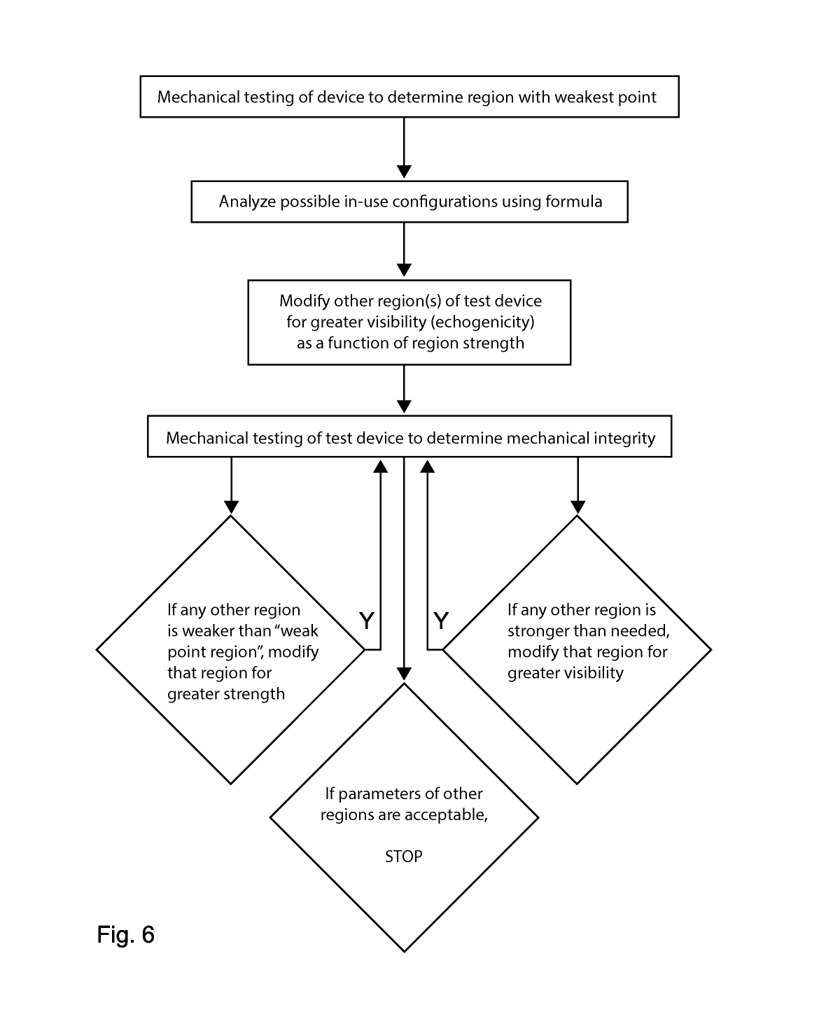

FIG. 6 is a flow chart showing the steps of a method that may be performed in the development of embodiments of the present invention. The first step is the mechanical testing of a device to determine the region with the weakest point. Such tests are known to a person having ordinary skill in the art. Examples are the three-point bend test, the tensile test, cantilever-style bending strength test and yield to failure test. The method of testing depends on the factor being investigated and the structure of the device. In general, such tests may be used to determine mechanical integrity.

The second step is to further analyze possible in-use configurations using formulas known to a person having ordinary skill in the art. Examples are the formulas previously disclosed in this description.

The third step is to modify region(s) of a test device not having the weakest point to increase visibility (e.g. echogenicity) as a function of the mechanical strength of the region(s) taking into account that the visibility features of concern reduce mechanical integrity. In other words, an echogenic feature may be added at a location of the medical device having greater mechanical integrity relative to other portions of the device.

The fourth step is mechanical testing of the test device to determine mechanical integrity of the modified test device.

The next step depends on the results of the previous mechanical testing. For example, if any other region is weaker than the “weak point region” (the region or portion of the device with the weakest point), then that region is modified to have greater structural strength. If a region is stronger than required, the design may be modified for that region to have greater visibility.

In the last step, if the parameters of the regions are found to be acceptable in the mechanical testing, the design process may be stopped, temporarily or permanently. A person having ordinary skill in the art would understand that further testing of the medical device for visibility under imaging may be required.

### Alternative Embodiments

In alternative embodiments, cuts or markings in the surface of a medical device may comprise, for example, dimples, notches (triangular in cross-section) or non-rounded grooves. The markings may be identical throughout region(s) of a device to facilitate manufacturing simplicity. Alternatively, the markings may vary in a specific region or throughout a device. For example, in an embodiment having cut away rings, the size of the rings could increase with each successive ring.

In some embodiments, as described hereinabove, the cuts or markings circumscribe the device substantially completely. In alternative embodiments, the cuts or markings are not formed around the entire circumference (or perimeter, for non-circular device cross-sections).

In general, an aspect of some embodiments of the invention is that larger volumes of cuts are made in areas of relatively lower expected mechanical stress; including wider cuts or markings, deeper cuts, and/or increased densities of cuts (more cuts per area).

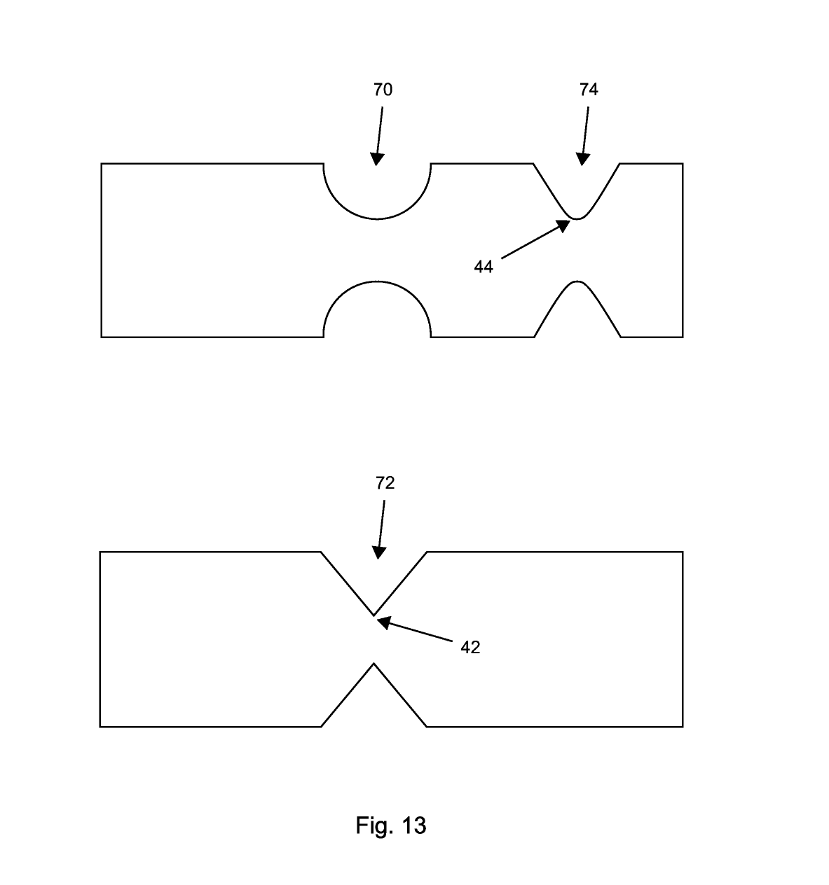

Some alternative embodiments include cuts with substantially sharp angles. As is understood by a person having ordinary skill in the art, a cut with sharp angles (i.e. sharp corners) is more susceptible to mechanical failure than a cut with rounded corners. The top illustration of FIG. 13 shows a rounded cut 70 (i.e. an arc-shaped cut). The lower illustration of FIG. 13 illustrates the example of a V-shaped cut 72 with a sharp angled cut 42 at the bottom of the “V” (i.e. a triangular-shaped cut). Sharp angled cut 42 will likely be susceptible to failure, as may be tested for using known means. In comparison, if a generally V-shaped surface cut is made with a rounded corner 44 at the bottom of the “V”, such as in rounded-V cut 74 (top right of FIG. 13), the corner will be less susceptible to failure than a sharp angled corner. Consequently, the shapes and angles of the cuts may be taken into account when considering the mechanical integrity of a medical device.

In alternative embodiments of the medical device, the echogenic feature comprises a textured surface, for example, a surface formed by grit blasting. In such embodiments, the echogenic feature scale may refer to, for example, the roughness of the surface, such that varying the echogenic feature scale along the device would involve varying the roughness of the texture at different locations along the device.

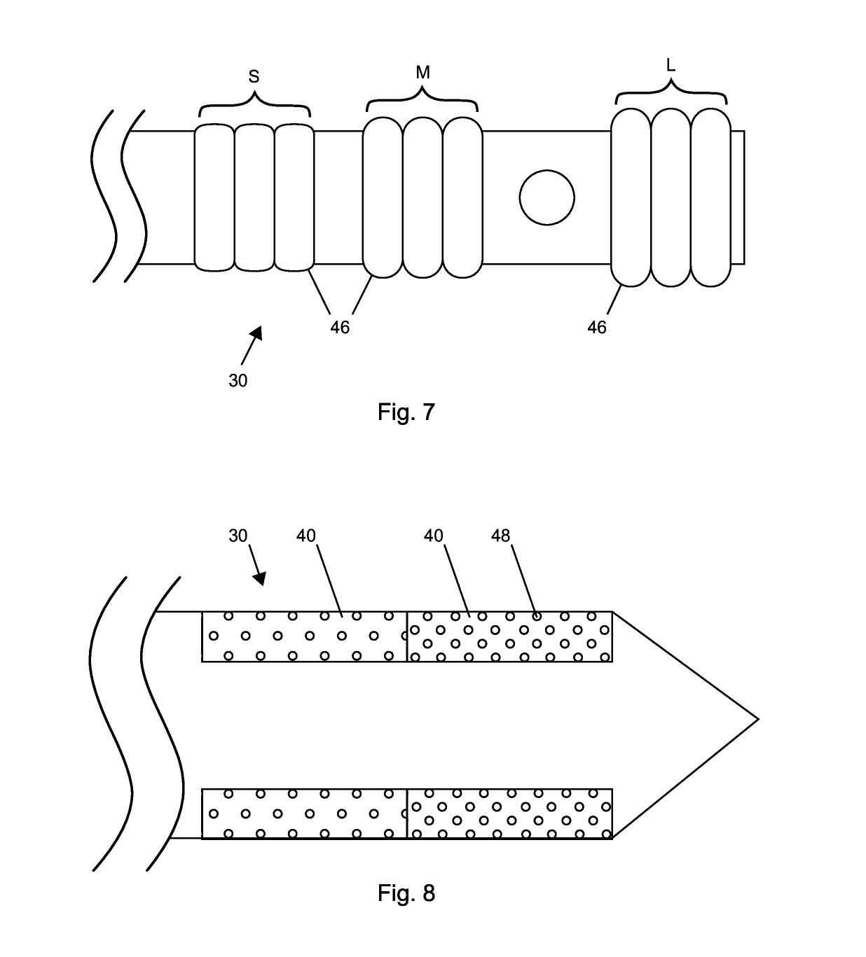

Embodiments of medical devices having echogenic features as described hereinabove include devices with features formed by removing material (e.g. FIGS. 1 to 4). In alternative embodiments, additive processes may be used to texturize the surface and form echogenic features such as, for example, bumps and/or projecting rings. Adding a material to a medical device by a process that involves heating the device may compromise the structural integrity of the device. FIG. 7 is a side view of the distal end of an embodiment of a medical device 30 having projecting rings 46. FIG. 7 illustrates three sizes of rings: small (S), medium (M) and large (L), with the small and medium rings proximal of the aperture. In some alternative embodiments, all of the rings may be the same size, or in other alternative embodiments, each successive projecting ring may vary in size.

If, in the design of a medical device, strong, homogenous material is removed to make space for a less strong but more visible material, the overall mechanical integrity at that location may be compromised. In one example, some of a device's metallic elongate member (a shaft) may be replaced, at locations of greater mechanical integrity (i.e. where the expected in-use stresses are lower), with an impregnated polymer that is structurally weaker than the metal, while the device size (e.g. outer diameter) is kept substantially constant. In a specific example, a device is modified to have about 50% of a metal sidewall, over a selected region, replaced by a polymer that contains bubbles or other echogenic features, without substantially changing the outer diameter of the device. In such and similar embodiments, the echogenic feature scale may refer to the amount of stronger material removed from the device.

An example of such an alternative embodiment is found in the FIG. 8 side view of the distal end of a medical device 30 having polymer coating 40 with trapped bubbles 48. The bubbles may be, for example, air pockets inside a porous material or gas inside hollow glass microspheres. In the embodiment of FIG. 8, there are two distinct regions having different concentrations of bubbles. In alternative embodiments, there may be a gradual change in bubble concentration. Bubble concentration may vary as a function of bubble size and/or density (i.e. the number of bubbles per a volume). The echogenic feature scale, in such an embodiment, may refer to the size and/or volume of bubbles, whereby the echogenic feature scale would vary along the device.

A partial list of materials and items that a polymer may contain to improve echogenicity includes, but is not limited to:

1) Sonically reflective particles wherein the sonically reflective material is selected from the group consisting of metal oxide powders, hollow glass microspheres and various forms of carbon particles;
2) Metal particles (tungsten, palladium, gold, silver, iron oxide, etc.);
3) Trapped bubbles; and
4) A braid, a strand or a strip of non-metal and biocompatible echogenic material.

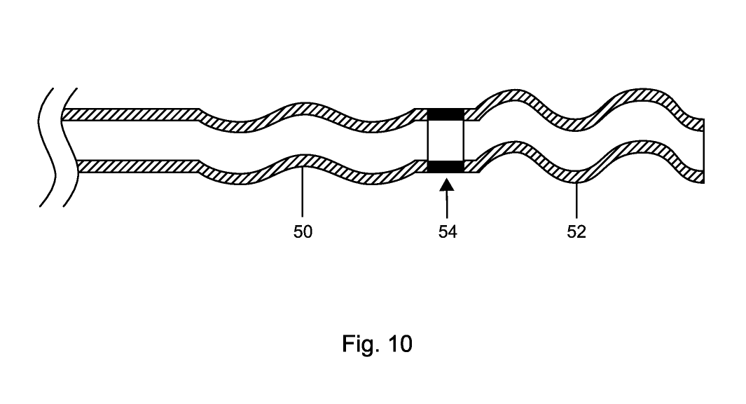

FIG. 10 illustrates an alternative embodiment comprising a medical device with curved surfaces 50, 52 and a radiopaque marker band 54. The curved surfaces 50, 52 are an echogenic feature that has been formed on the outside surface of the device without adding or subtracting material. In some embodiments, the material is heat-treated to form the curved surfaces. Heat-treating a device's material may reduce the mechanical integrity of the device. In other embodiments, the material of a device is worked when cold to give the device curved surfaces (i.e. the surfaces are formed by mechanical deformation). In some particular embodiments, there is a change of diameter along the elongate member (shaft) of the device. Mechanical deformation of a device when the device is cold may also reduce the mechanical integrity of the device, particularly in the areas being deformed. When an embodiment with cut-out rings also includes a region that has been heated, or has a change of diameter, the heated region or the diameter change is a factor in determining the location and size of the rings.

The embodiment of FIG. 10 also includes radiopaque marker band 54 for X-ray imaging. Using, for example, welding (heating) or crimping (deformation) to attach radiopaque or other components can further reduce device integrity. In the embodiment shown in FIG. 10, the portion of the device that has the radiopaque marker has does not have the curved surfaces 50, 52. In such embodiments, the echogenic feature scale may refer to the curvature of the curved portions, the depth of the curve, the size of the radiopaque marker (or other attached component), etc. Any or all of these features may vary along the device dependent upon the expected mechanical stresses at any particular location along the device, as described hereinabove.

In general, a non-uniform characteristic (distribution, size, configuration, etc.) of a feature enhancing visibility under imaging may be a function of local stresses. Specifically, denser patterns of visibility enhancing features and/or larger visibility enhancing features may be located in areas of expected (relatively) low mechanical stress.

Embodiments of the present invention avoid adding excess visibility enhancing features that reduce mechanical integrity at locations already having greater mechanical vulnerability, either due to their location along the device or due to other features existing at those locations which reduce mechanical integrity. Features that reduce the mechanical integrity (i.e. increase mechanical vulnerability) of a device may be referred to as mechanical discontinuities. A partial list of such features includes, but is not limited to, joints, apertures (including side-ports or lateral apertures), handles, proximal supports, hinges, and welds, material deformed by crimping, locations subject to heat during manufacture or use, and other features interrupting device structural continuity.

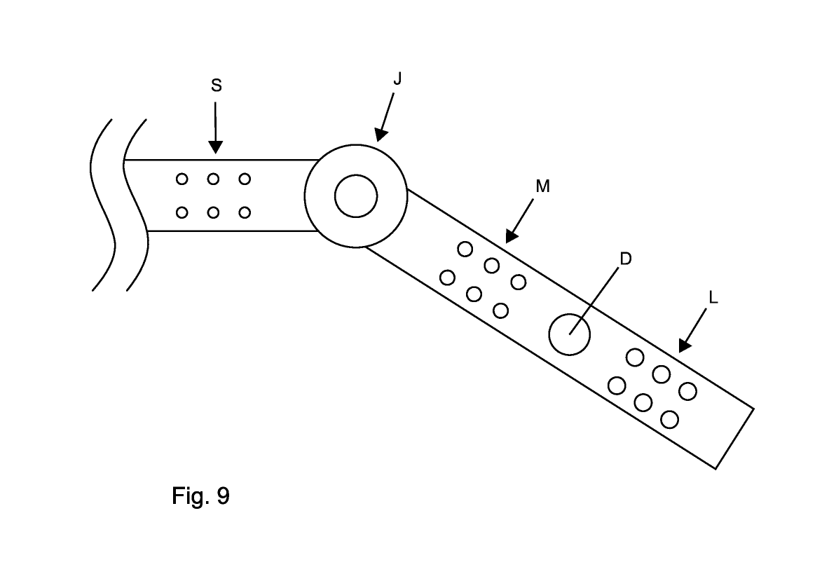

FIG. 9 is a side view of the distal end of an embodiment of a medical device having structural/mechanical discontinuities. The FIG. 9 embodiment includes a hinged joint J and discontinuity D, which may be an aperture or a radiopaque marker. The visibility enhancing feature (a plurality of dimples) is located in regions between the structural discontinuities. There are three sizes of dimples, small, medium and large, labeled as S, M and L in FIG. 9. The portions of the device having the mechanical discontinuities lack the visibility enhancing feature and the size of the dimples (i.e. the echogenic feature scale for such an embodiment) varies along the device.

### Use of Embodiments of the Present Invention

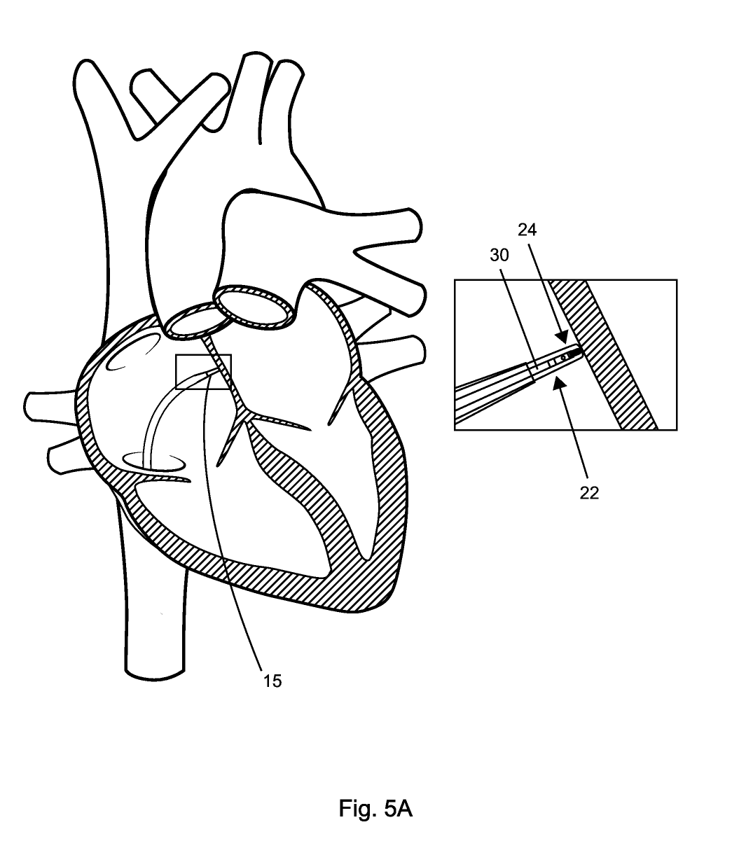

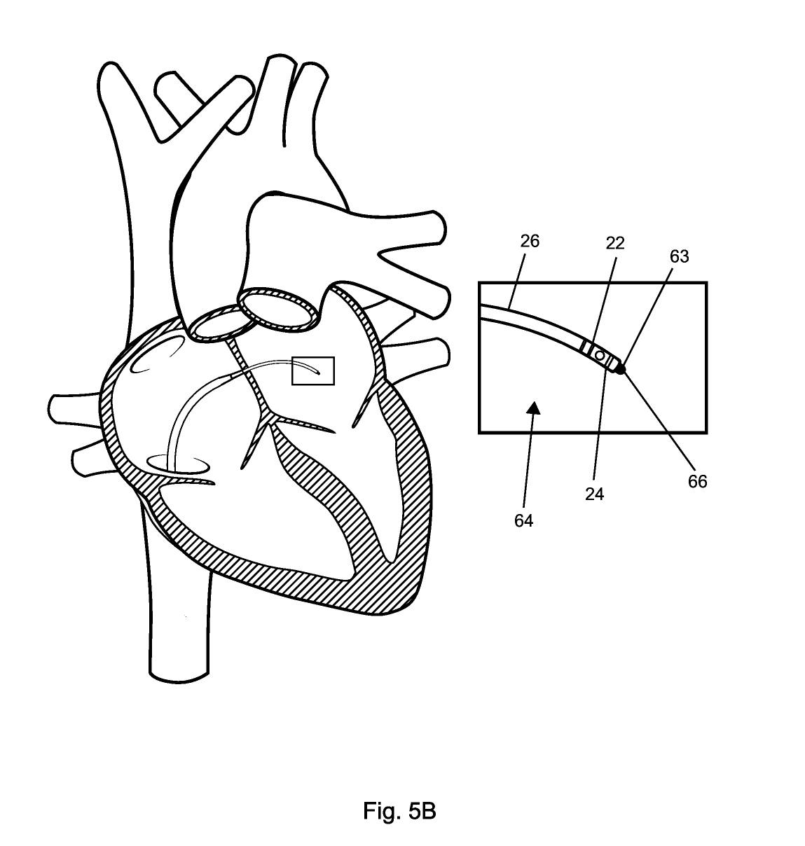

One specific embodiment is for a method of using a medical device as disclosed herein, for example as illustrated in FIGS. 5A and 5B. The target site for use may comprise a tissue within the heart of a patient, for example the atrial septum of the heart. In such an embodiment, the target site may be accessed via the inferior vena cava (IVC), for example through the femoral vein, with said access being facilitated by imaging of radiopaque marker 66 of functional tip 15 and echogenic cut-out rings 22 and 24 of distal portion 64 during advancement of medical device 30 (a needle/radiofrequency perforation apparatus). This embodiment includes providing a medical device 30 comprising a functional tip 15 that is visible under fluoroscopic imaging so as to be visibly distinct from the rest of the medical device and small cut-out rings 22 (proximal of aperture 20) and large cut-out rings 24 (distal of aperture 20) that are visible under an ultrasound imaging system. Intracardiac echocardiography (ICE) or transesophageal echocardiography (TEE) ultrasound techniques may be used.

In one such embodiment, an intended user introduces a guidewire into a femoral vein, typically the right femoral vein, and advances it towards the heart. A guiding sheath, for example a sheath as described in U.S. patent application Ser. No. 10/666,288 (filed on Sep. 19, 2003), incorporated herein by reference, is then introduced into the femoral vein over the guidewire, and advanced towards the heart. The distal ends of the guidewire and sheath are then positioned in the superior vena cava. These steps may be performed with the aid of an imaging system appropriate for radiopaque marker 66 and an ultrasound imaging system appropriate for echogenic cut-out rings 22 and 24. When the sheath is in position, a dilator, for example the TorFlex™ Transseptal Dilator of Baylis Medical Company Inc. (Montreal, Canada), or the dilator as described in U.S. patent application Ser. No. 11/727,302 (filed on Mar. 26, 2007), incorporated herein by reference, is introduced into the sheath and over the guidewire, and advanced through the sheath into the superior vena cava. Alternatively, the dilator may be fully inserted into the sheath prior to entering the body, and both may be advanced simultaneously towards the heart. When the guidewire, sheath, and dilator have been positioned in the superior vena cava, the guidewire is removed from the body, and an electrosurgical device, for example medical device 30 (a radiofrequency perforation apparatus), is then introduced into the lumen of the dilator, and advanced toward the heart. The sheath, dilator and medical device 30 are retracted slightly, such that they enter the right atrium of the heart.

In this embodiment, after inserting the electrosurgical device into a dilator, the user may position the distal end of the dilator against the atrial septum. The electrosurgical device is then positioned using imaging of a marker 66 of functional tip 15 such that electrode 63 (which also functions as a radiopaque marker) is aligned with or protruding slightly from the distal end of the dilator but not retracted inside of the dilator. The dilator and medical device 30 are dragged along the atrial septum and positioned, for example against the fossa ovalis of the atrial septum utilizing medical imaging techniques. Small cut-out rings 22 (proximal of aperture 20) and large cut-out rings 24 (distal of aperture 20), visible under an ultrasound imaging system, may aid in positioning and orientating functional tip 15. A variety of additional steps may be performed, such as measuring one or more properties of the target site, for example an electrogram or ECG (electrocardiogram) tracing and/or a pressure measurement or delivering material to the target site, for example delivering a contrast agent through aperture(s) 20. Such steps may facilitate the localization of the electrode 63 at the desired target site. In addition, tactile feedback provided by medical device 30 (a radiofrequency perforation apparatus) is usable to facilitate positioning of the electrode 63 at the desired target site. The practitioner can visually monitor the position of functional tip 15 as it is advanced upwards into the heart and as it is dragged along the surface of the atrial septum and positioned in the groove of the fossa ovalis using one or more of the radiopaque marker and echogenic rings.

With the electrosurgical device and the dilator positioned at the target site, energy is delivered from the energy source, through medical device 30 (a radiofrequency perforation apparatus), to the target site. For example, if the medical device 30 is used, electrical energy is delivered through the elongate member 26, to the electrode 63, and into the tissue at the target site. In some embodiments, the electrical energy is delivered at a power of at least about 5 W at a voltage of at least about 75 V (peak-to-peak), and functions to vaporize cells in the vicinity of the electrode, thereby creating a void or perforation through the tissue at the target site. Typically, energy is delivered in the radiofrequency range. If the heart was approached via the inferior vena cava, as described hereinabove, the user applies force in the substantially cranial direction to the handle 1 of the electrosurgical device as energy is being delivered. The force is then transmitted from the handle to the distal portion 64 of the medical device 30, such that the distal portion 64 of elongate member 26 advances at least partially through the perforation. In these embodiments, when the distal portion 64 has passed through the target tissue, that is, when it has reached the left atrium, energy delivery is stopped/terminated. In some embodiments, the step of delivering energy occurs over a period of between about 1 s and about 5 s.

Further details regarding fluoroscopic imaging and radiopaque markers may be found in U.S. provisional application 61/653,967, filed May 31, 2012, incorporated herein by reference.

In the method described hereinabove, echogenic features such as those described hereinabove facilitate the localization and/or navigation of the medical device, prior to, during and after the medical procedure. Providing such visibility enhancing features while minimizing the effect on the mechanical integrity of the device, for example by employing a varying echogenic feature scale as described hereinabove, is particularly beneficial in such procedures which may utilize ultrasonic imaging for guidance and localization and which may involve mechanical forces being applied to the device.

Thus, as described herein, the present inventors have discovered and reduced to practice a novel and unique medical device including a means for enhancing visibility under imaging (a visibility enhancing feature) which, while reducing the local mechanical strength, avoids compromising the overall device effectiveness or safety. Embodiments of the device provide increased visibility along the device both in areas of relatively greater mechanical vulnerability and lesser mechanical vulnerability, wherein the visibility is increased to a greater extent in areas of lesser mechanical vulnerability relative to areas of greater mechanical vulnerability. In other words, visibility under imaging is increased by a non-uniform distribution of visibility-enhancing features as a function of the local stresses expected during use.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A medical device comprising an elongate member having proximal and distal regions, the distal region comprising:
a first echogenic region including a plurality of circumferential first region cuts, each of the first region cuts having a first region cut volume; and
a second echogenic region including a plurality of circumferential second region cuts, each of the second region cuts having a second region cut volume greater than said first region cut volume;
wherein the second echogenic region is located at a portion of the distal region subject to less bending stress in use, relative to a location of the first echogenic region.

2. The medical device of claim 1, the elongate member having a circular cross-section, the elongate member being operable to be inserted through a dilator when in use, the elongate member configured such that, in use, the second echogenic region is located further distal than the first echogenic region relative to an end of the dilator through which the elongate member is inserted.

3. The medical device of claim 1, wherein mechanical integrity of the elongate member under stress is substantially equivalent along its length.

4. The medical device of claim 1, wherein each of the second region cuts has a depth of between 33% and 50% of a nominal wall thickness of the elongate member.

5. The medical device of claim 1, wherein each of the first region cuts has a depth of between about 13% and about 27% of a nominal wall thickness of the elongate member.

6. The medical device of claim 1, wherein each of the second region cuts has a second region cut width and each of said first region cuts has a first region cut width, wherein the second region cut width is greater than the first region cut width.

7. The medical device of claim 1, wherein each of the second region cuts has a second region cut depth and each of said first region cuts has a first region cut depth, wherein the second region cut depth is greater than the first region cut depth.

8. The medical device of claim 1, wherein a second region cut density is greater than a first region cut density.

9. The medical device of claim 1, wherein the elongate member defines a side-port through a wall of the elongate member, and wherein the first echogenic region is proximal of the side-port and the second echogenic region is distal of the side-port.

10. The medical device of claim 1 wherein at least one of the first region cuts or second region cuts define a cut selected from the group consisting of an arc-shaped cut, a triangular-shaped cut and a v-shaped cut.

11. The medical device of claim 1, further comprising a coating covering the elongate member, the coating having an acoustic impedance from 1.3 MRayl to 1.7 MRayl.

12. The medical device of claim 11, wherein the coating comprises an epoxy resin.

13. A medical device comprising an elongate member having proximal and distal regions, the distal region comprising:
a first echogenic region including a plurality of circumferential first region cuts, each of the first region cuts having a first region cut volume; and
a second echogenic region, distal of the first echogenic region, including a plurality of circumferential second region cuts, each of the second region cuts having a second region cut volume greater than said first region cut volume.

14. The medical device of claim 13, the elongate member having a circular cross-section, the elongate member being operable to be inserted through a dilator when in use, whereby the second echogenic region is located further distal than the first echogenic region relative to an end of the dilator through which the elongate member is inserted.

15. The medical device of claim 13, wherein mechanical integrity of the elongate member under stress is substantially equivalent along its length.

16. The medical device of claim 13, wherein each of the first region cuts and each of the second region cuts are formed around substantially 360° of a circumference of the medical device.

17. A method of creating a perforation of tissue within a heart, the method comprising:
inserting an elongate medical device into a heart of a patient's body;
visualizing one or more echogenic markings associated with the medical device to facilitate positioning of the medical device at a target tissue site within the heart; and
delivering energy to a distal region of the elongate medical device to create a perforation through the target tissue site.

18. The method of claim 17, wherein the target tissue site comprises a septum of a heart.

19. The method of claim 17, wherein delivering energy comprises delivering electrical energy in a radiofrequency range.

20. The method of claim 18, further comprising a step of advancing a distal portion of the medical device through the perforation of the septum and into a left atrium of the heart.

21. The method of claim 20, wherein the step of advancing the distal portion of the medical device comprises visualizing the one or more echogenic markings associated with the medical device while advancing the distal portion of the medical device through the perforation.

22. The method of claim 21, further comprising a step of visualizing the one or more echogenic markings associated with the medical device following the step of advancing the distal portion of the medical device, to confirm the position of the medical device.
